(19) United States
(12) Patent Application Publication
(10) Pub. No.: US 2023/0097431 A1
(43) Pub. Date: Mar. 30, 2023

(54) Medical Instrument Shape Filtering Systems and Methods

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/952,645

(22) Filed: Sep. 26, 2022

Related U.S. Application Data
(60) Provisional application No. 63/248,917, filed on Sep. 27, 2021.

(51) Int. Cl. A61B 34/20

(52) U.S. Cl. CPC A61B 34/20; A61B 2034/2061; A61B 2034/2055

(57) ABSTRACT

Disclosed herein is a medical device system and method for detecting placement of a medical device having an elongate probe for insertion within a patient body. The system can determine a live 3D shape of the elongate probe via shape sensing of an optical fiber extending along the elongate probe during insertion. The system can capture a reference shape of the live 3D shape and define a pathway in front of the live 3D shape. A user can be notified when the live 3D shape exceeds a buffer zone of the pathway. A current reference shape can be compared with a preceding reference frame to assess a validity of the current reference frame.

### PRIORITY

This application claims the benefit of priority to U.S. Provisional Application No. 63/248,917, filed Sep. 27, 2021, which is incorporated by reference in its entirety into this application.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving stylets. Generally, electromagnetic tracking systems feature three components: a field generator, a sensor unit and control unit. The field generator uses several coils to generate a position-varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Although electromagnetic tracking systems avoid line-of-sight reliance in tracking the tip of a stylet while obviating radiation exposure and potentially harmful contrast media associated with fluoroscopic methods, electromagnetic tracking systems are prone to interference. More specifically, since electromagnetic tracking systems depend on the measurement of magnetic fields produced by the field generator, these systems are subject to electromagnetic field interference, which may be caused by the presence of many different types of consumer electronics such as cellular telephones. Additionally, electromagnetic tracking systems are subject to signal drop out, depend on an external sensor, and are defined to a limited depth range.

Disclosed herein is a fiber optic shape sensing system and methods performed thereby where the system is configured to determine a three dimensional shape of the medical device equipped with an optical fiber during insertion within the patient and capture the three-dimensional shape as reference shape to be used in defining a pathway to serve as a guide for further insertion of the medical device.

### SUMMARY

Briefly summarized, disclosed herein a medical device system for detecting placement of a medical device within a patient body, where the system includes the medical device and a console. The medical device includes an elongate probe and an optical fiber having one or more of core fibers extending along the elongate probe. Each of the one or more core fibers includes a plurality of sensors distributed along the longitudinal length and each sensor of the plurality of sensors is configured to (i) reflect a light signal of a different spectral width based on received incident light, and (ii) change a characteristic of the reflected light signal based on strain experienced by the optical fiber.

The console includes one or more processors and a non-transitory computer-readable medium having stored thereon logic, when executed by the one or more processors, causes operations of the system. The operations include determining a live three-dimensional (3D) shape of the elongate probe during insertion of the elongate probe within the patient body, where determining includes (i) providing an incident light signal to the optical fiber, (ii) receiving reflected light signals of different spectral widths of the incident light by one or more of the plurality of sensors, and (iii) processing the reflected light signals associated with the one or more of core fibers to determine the live 3D shape.

The operations further include (i) capturing a reference shape, where the reference shape includes at least a portion of the live 3D shape, and (ii) defining a pathway for the live 3D shape, the pathway extending distally away from a distal end of the reference shape.

In some embodiments, the medical device is one of an intravascular device, an endoscope, a biopsy device, a drainage catheter, a surgery device, a tissue ablation device, or a kidney stone removal device.

The operations may further include (i) rendering an image of the reference shape on a display of the console, (ii) rendering an image of the pathway on the display, and/or rendering an image of the live 3D shape in combination with the image of the pathway on the display.

The operations may further include comparing the live 3D shape with the reference shape, and as a result of the comparison, detecting an insertion and/or withdrawal displacement of the elongate probe. The operations may also include capturing a plurality of reference shapes of the live 3D shape, and defining the pathway in accordance with the plurality of reference shapes. The operations may further include (i) defining a buffer zone for the live 3D shape, where the buffer zone extends radially away from the pathway, (ii) comparing the live 3D shape with the buffer zone, and (iii) as a result of the comparison, providing a notification when a portion of the live 3D shape exceeds the buffer zone.

In some embodiments, the system is communicatively coupled with an imaging system, and the operations further include receiving image data from the imaging system and defining the pathway in accordance with the image data. The imaging system may include one or more of an ultrasound imaging system, a magnetic resonance imaging (MM) system, a computed tomography (CT) imaging system, an X-ray system, an X-ray system including fluoroscopy, or an electro-anatomical mapping system.

The elongate probe may include one or more sensors configured to detect physiological conditions of the patient, and the operations may further include defining the pathway in accordance with sensor data pertaining to the physiological conditions. The physiological conditions may include one or more of a body temperature, a fluid pressure, a blood flow rate, or an ECG signal.

In some embodiments, the operations include defining the pathway in accordance with one or more reference shapes captured during the insertion of previous elongate probes.

Also disclosed herein is a method for detecting placement of a medical device within a patient body, where the method includes (i) providing the medical device coupled with a medical device system, the medical device including an elongate probe configured for insertion within the patient body, (ii) determining a live three-dimensional (3D) shape of the elongate probe inserted within the patient body.

Determining the live three-dimensional (3D) shape includes providing an incident light signal to an optical fiber extending along the elongate probe, where the optical fiber includes a one or more of core fibers. Each of the one or more of core fibers includes a plurality of reflective gratings distributed along a longitudinal length of a corresponding core fiber and each of the plurality of reflective gratings is configured to (i) reflect a light signal of a different spectral width based on received incident light, and (ii) change a characteristic of the reflected light signal based on strain experienced by the optical fiber. Determining the live three-dimensional (3D) shape further includes receiving reflected light signals of different spectral widths of the incident light by one or more of the plurality of sensors and processing the reflected light signals associated with the one or more of core fibers to determine the three-dimensional shape of the elongate probe inserted within the patient body.

The method further includes (i) capturing a reference shape, the reference shape including at least a portion of the live 3D shape and (ii) defining a pathway for the live 3D shape, the pathway extending distally away from a distal end of the reference shape.

The method may further include (i) rendering an image of the reference shape on a display of the console, (ii) rendering an image of the pathway on the display, and/or rendering an image of the live 3D shape in combination with the image of the pathway on the display.

The method may further include comparing the live 3D shape with the reference shape, and as a result of the comparison, detecting an insertion and/or withdrawal displacement of the elongate probe. The method may also include capturing a plurality of reference shapes of the live 3D shape, and defining the pathway in accordance with the plurality of reference shapes. The method may further include (i) defining a buffer zone for the live 3D shape, where the buffer zone extends radially away from the pathway, (ii) comparing the live 3D shape with the buffer zone, and as a result of the comparison, providing a notification when a portion of the live 3D shape exceeds the buffer zone.

The method may further include (i) coupling the medical device system with an imaging system, (ii) receiving image data from the imaging system, and (iii) defining the pathway in accordance with the image data. The imaging system may include one or more of an ultrasound imaging system, a magnetic resonance imaging (MRI) system, a computed tomography (CT) imaging system, an X-ray system, an X-ray system including fluoroscopy, or an electro-anatomical mapping system.

In some embodiments of the method, the elongate probe includes one or more sensors configured to detect physiological conditions of the patient, and the method further includes defining the pathway in accordance with sensor data pertaining to the physiological conditions. The physiological conditions may include one or more of a body temperature, a blood pressure, a blood flow rate, or an ECG signal.

The method may further include defining the pathway in accordance with reference shapes captured during the insertion of previous elongate probes.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustrative embodiment of a medical instrument monitoring system including a medical instrument with optic shape sensing capabilities, in accordance with some embodiments;

FIG. 2 is an exemplary embodiment of a structure of a section of the multi-core optical fiber included within the elongate probe of FIG. 1, in accordance with some embodiments;

FIG. 3A is a first exemplary embodiment of the probe of FIG. 1 supporting both an optical and electrical signaling, in accordance with some embodiments;

FIG. 3B is a cross sectional view of the probe of FIG. 3A, in accordance with some embodiments;

FIG. 4A is a second exemplary embodiment of the probe of FIG. 1, in accordance with some embodiments;

FIG. 4B is a cross sectional view of the probe of FIG. 4A, in accordance with some embodiments;

FIG. 5A is an elevation view of a first illustrative embodiment of a catheter including integrated tubing, a diametrically disposed septum, and micro-lumens formed within the tubing and septum, in accordance with some embodiments;

FIG. 5B is a perspective view of the first illustrative embodiment of the catheter of FIG. 5A including core fibers installed within the micro-lumens, in accordance with some embodiments;

FIGS. 6A-6B are flowcharts of the methods of operations conducted by the medical instrument monitoring system of FIG. 1 to achieve optic three-dimensional shape sensing in accordance with some embodiments;

FIG. 7 is an illustration of an exemplary implementation of the system of FIG. 1, in accordance with some embodiments;

FIGS. 8A-8B illustrate the 3D shape of FIG. 7 of the elongate probe at sequential states of insertion, in accordance with some embodiments;

FIG. 8C illustrates an exemplary instance of insertion of the probe in contrast to the state of insertion of FIG. 8B, where the live 3D shape deviates from the pathway, in accordance with some embodiments;

FIG. 8D illustrates a screen shot of the live 3D shape of FIG. 7 as may be rendered on the display, in accordance with some embodiments; and

FIG. 9 illustrates a flowchart of a method of operations as may be performed by the medical instrument monitoring system of FIG. 1 to define a pathway for the live 3D shape of the elongate probe, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

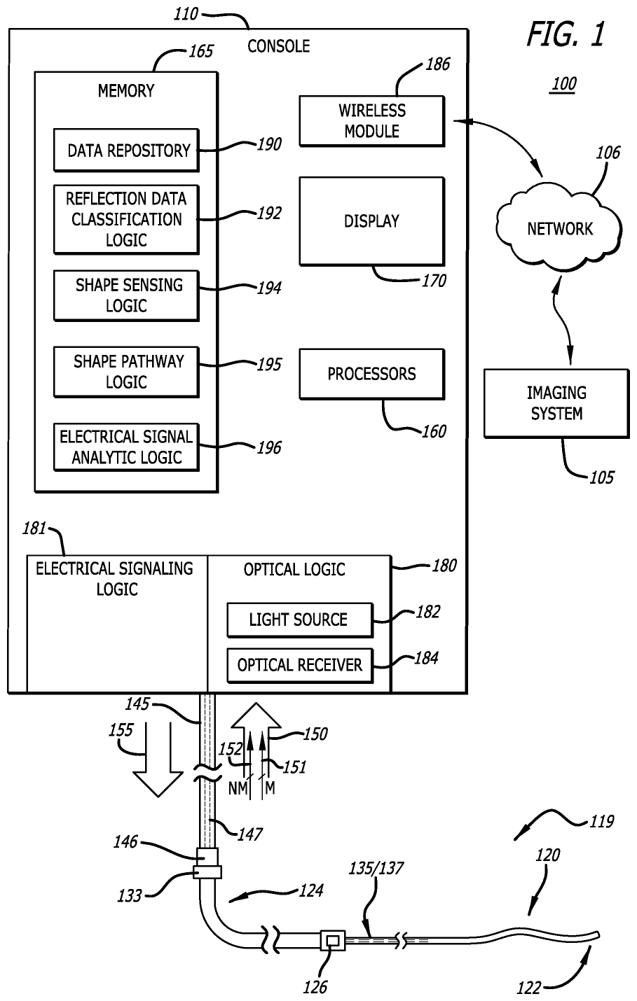

FIG. 1 illustrates an embodiment of a medical instrument placement system including a medical instrument. As shown, the medical instrument placement system (system) 100 generally includes a console 110 and a medical instrument 119 communicatively coupled with the console 110. For this embodiment, the medical instrument 119 includes an elongate probe 120 defining a distal end 122 and having a console connector 133 on a proximal end 124. The elongate probe 120 includes an optical fiber 135 extending along a length of the elongate probe 120 as further described below. The console connector 133 enables the medical instrument 119 to be operably connected to the console 110 via an interconnect 145 including one or more optical fibers 147 (hereinafter, “optical fiber(s)”) and a conductive medium terminated by a single optical/electric connector 146 (or terminated by dual connectors). Herein, the connector 146 is configured to engage (mate) with the console connector 133 to allow for the propagation of light between the console 110 and the medical instrument 119 as well as the propagation of electrical signals from the elongate probe 120 to the console 110.

The medical instrument 119 including the elongate probe 120 may be configured to perform any of a variety of medical procedures. As such, the medical instrument 119 may be a component of or employed with a variety of medical devices. In some implementations, the medical instrument 119 may take the form of a guidewire or a stylet for employment with a catheter, for example. In some implementations, the medical instrument 119 may be integrated into an endoscope. Other exemplary implementations include drainage catheters, surgery devices, stent insertion and/or removal devices, biopsy devices, and kidney stone removal devices. In short, the medical instrument 119 may be employed with, or the elongate probe 120 may be a component of, any medical device that is inserted into a patient.

According to one embodiment, the console 110 includes one or more processors 160, a memory 165, a display 170, and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The one or more processors 160, with access to the memory 165 (e.g., non-volatile memory or non-transitory, computer-readable medium), are included to control functionality of the console 110 during operation. As shown, the display 170 may be a liquid crystal diode (LCD) display integrated into the console 110 and employed as a user interface to display information to the clinician, especially during an instrument placement procedure. In another embodiment, the display 170 may be separate from the console 110. Although not shown, a user interface is configured to provide user control of the console 110.

In some implementations, the console 110 may be communicatively coupled with an imaging system(s) 105 which may include one or more of an ultrasound imaging system, a magnetic resonance imaging (MRI) system, a computed tomography (CT) imaging system, an X-ray system, an X-ray system including fluoroscopy, or an electro-anatomical mapping system. As such, in some embodiments, the console 110 includes a wireless module 186 for facilitating communication with the imaging system 105. The imaging system 105 may be communicatively coupled with the console 110 via a wireless communication protocol across a network 106. In other embodiments, the imaging system 105 may be integrated into the system 100 (or more specifically the console 110) or coupled with the console via a wired connection.

With further reference to FIG. 1, a first architecture of the system 100 is illustrated. The architecture of the system 100 may include the console 110, the imaging system 105, and a network 106. In some embodiments, the imaging system 105 may include more one than imaging system 105. The network 106 represents the communication pathways between the console 110 and the imaging system 105. In one embodiment, the network 106 is the Internet. The network 106 can also utilize dedicated or private communication links (e.g., WAN, MAN, or LAN) that are not necessarily part of the Internet. The network 106 may use standard communications technologies and/or protocols.

The shape pathway (or filtering) logic 195 may receive and process data from the imaging system 105 as further described below. The shape pathway logic 195 may be in the form of a software application that is loaded on the console 110 and executable by the one or more processors 160. In other embodiments, the shape pathway logic 195 need not be loaded on the console 110 but may instead execute within a cloud computing environment (which may also be represented by the reference numeral 106) such that data from the data repository 190 as well as data from the imaging system 105 are communicated to shape pathway logic 195 for processing. Thus, any shape pathway logic 195 represented as being part of the console 110 may include an application programming interface (API) that is configured to transmit and receive data communication messages to and from the shape pathway logic 195 operating in the cloud computing environment.

According to the illustrated embodiment, the content depicted by the display 170 may change according to which mode the elongate probe 120 is configured to operate: optical, TLS, ECG, or another modality. In TLS mode, the content rendered by the display 170 may constitute a two-dimensional or three-dimensional representation of the physical state (e.g., length, shape, form, and/or orientation) of the elongate probe 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

According to one embodiment of the disclosure, an activation control 126, included on the medical instrument 119, may be used to set the elongate probe 120 into a desired operating mode and selectively alter operability of the display 170 by the clinician to assist in medical device placement. For example, based on the modality of the elongate probe 120, the display 170 of the console 110 can be employed for optical modality-based guidance during probe advancement through the vasculature or TLS modality to determine the physical state (e.g., length, form, shape, orientation, etc.) of the elongate probe 120. In one embodiment, information from multiple modes, such as optical, TLS or ECG for example, may be displayed concurrently (e.g., at least partially overlapping in time).

Referring still to FIG. 1, the optical logic 180 is configured to support operability of the medical instrument 119 and enable the return of information to the console 110, which may be used to determine the physical state associated with the elongate probe 120 along with monitored electrical signals such as ECG signaling via an electrical signaling logic 181 that supports receipt and processing of the received electrical signals from the elongate probe 120, (e.g., ports, analog-to-digital conversion logic, etc.). The physical state of the elongate probe 120 may be based on changes in characteristics of the reflected light signals 150 received at the console 110 from the elongate probe 120. The characteristics may include shifts in wavelength caused by strain on certain regions of the core fibers integrated within the optical fiber core 135 positioned within or operating as the elongate probe 120, as shown below. As discussed herein, the optical fiber core 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the elongate probe 120.

According to one embodiment of the disclosure, as shown in FIG. 1, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the elongate probe 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the elongate probe 120, and (ii) translate the reflected light signals 150 into reflection data (from a data repository 190), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the one or more processors 160, which governs their operation. Also, the optical receiver 184 is operably coupled to provide the reflection data (from the data repository 190) to the memory 165 for storage and processing by reflection data classification logic 192. The reflection data classification logic 192 may be configured to: (i) identify which core fibers pertain to which of the received reflection data (from the data repository 190) and (ii) segregate the reflection data stored within the data repository 190 provided from reflected light signals 150 pertaining to similar regions of the elongate probe 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the elongate probe 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in three-dimensional space and may further determine the current physical state of the elongate probe 120 in three-dimensional space for rendering on the display 170.

According to one embodiment of the disclosure, the shape sensing logic 194 may generate a rendering of the current physical state of the elongate probe 120, based on heuristics or run-time analytics. For example, the shape sensing logic 194 may be configured in accordance with machine-learning techniques to access the data repository 190 with pre-stored data (e.g., images, etc.) pertaining to different regions of the elongate probe 120 in which reflected light from core fibers have previously experienced similar or identical wavelength shifts. From the pre-stored data, the current physical state of the elongate probe 120 may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the elongate probe 120, especially to enable guidance of the elongate probe 120 when positioned within the patient and at a desired destination within the body.

The console 110 may further include electrical signaling logic 181, which is positioned to receive one or more electrical signals from the elongate probe 120. The elongate probe 120 is configured to support both optical connectivity as well as electrical connectivity. The electrical signaling logic 181 receives the electrical signals (e.g., ECG signals) from the elongate probe 120 via the conductive medium. The electrical signals may be processed by electrical signal logic 196, executed by the one or more processors 160, to determine ECG waveforms for display.

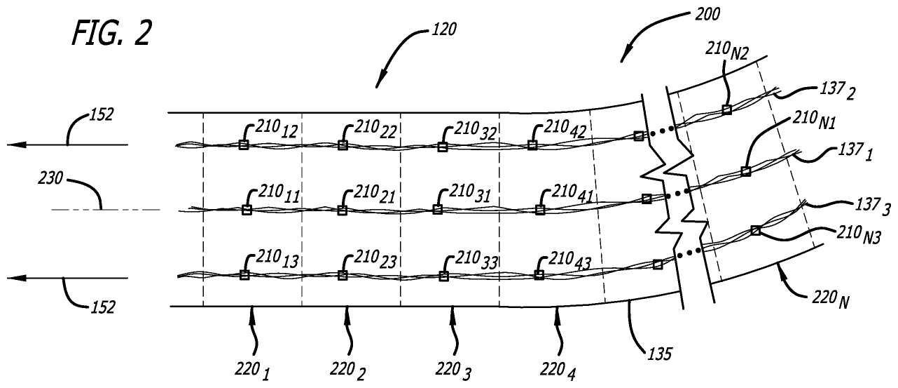

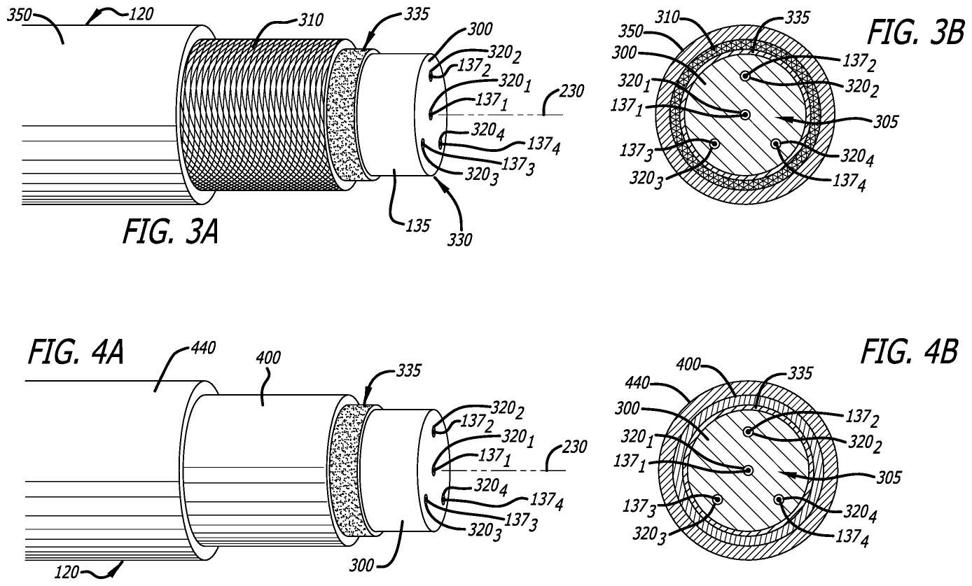

Referring to FIG. 2, an exemplary embodiment of a structure of a section of the multi-core optical fiber included within the elongate probe 120 of FIG. 1 is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the elongate probe 120 is operative, each of the reflective gratings 210₁-210_{N} reflects light for a different spectral width. As shown, each of the gratings 210₁ᵢ-210_{Ni} (1≤i≤M) is associated with a different, specific spectral width, which would be represented by different center frequencies of f ₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the elongate probe 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the elongate probe 120 advances in the patient.

For example, with respect to the multi-core optical fiber section 200 of FIG. 2, in response to angular (e.g., radial) movement of the elongate probe 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 1373 with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the elongate probe 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the elongate probe 120. The reflected light signals 150 are reflected back to the console 110 via individual paths over a particular core fiber 137₁-137_{M}.

Referring to FIG. 3A, a first exemplary embodiment of the stylet of FIG. 1 supporting both an optical and electrical signaling is shown in accordance with some embodiments. Herein, the elongate probe 120 features a centrally located multi-core optical fiber 135, which includes a cladding 300 and a plurality of core fibers 137₁-137_{M} (M≥2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the multi-core optical fiber 135 and the elongate probe 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the elongate probe 120, as a greater spacing may provide a lesser rigidity, and thereby, a more pliable elongate probe 120.

According to this embodiment of the disclosure, as shown in FIGS. 3A-3B, the core fibers 137₁-137₄ include (i) a central core fiber 1371 and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the elongate probe 120, the braided tubing 310 provides mechanical integrity to the multi-core optical fiber 135 and operates as a conductive pathway for electrical signals. For example, the braided tubing 310 may be exposed to a distal tip of the elongate probe 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1 is shown in accordance with some embodiments. Referring now to FIG. 4A, a second exemplary embodiment of the elongate probe 120 of FIG. 1 supporting both an optical and electrical signaling is shown. Herein, the elongate probe 120 features the multi-core optical fiber 135 described above and shown in FIG. 3A, which includes the cladding 300 and the first plurality of core fibers 137₁-137_{M} (M≥3; M=4 for embodiment) residing within the corresponding plurality of lumens 320₁-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the elongate probe 120 in the transfer of electrical signals (e.g., ECG signals) to the console, the conductive tubing 400 may be exposed up to a tip 410 of the elongate probe 120. For this embodiment of the disclosure, a conductive epoxy 420 (e.g., metal-based epoxy such as a silver epoxy) may be affixed to the tip 410 and similarly joined with a termination/connection point created at a proximal end 430 of the elongate probe 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

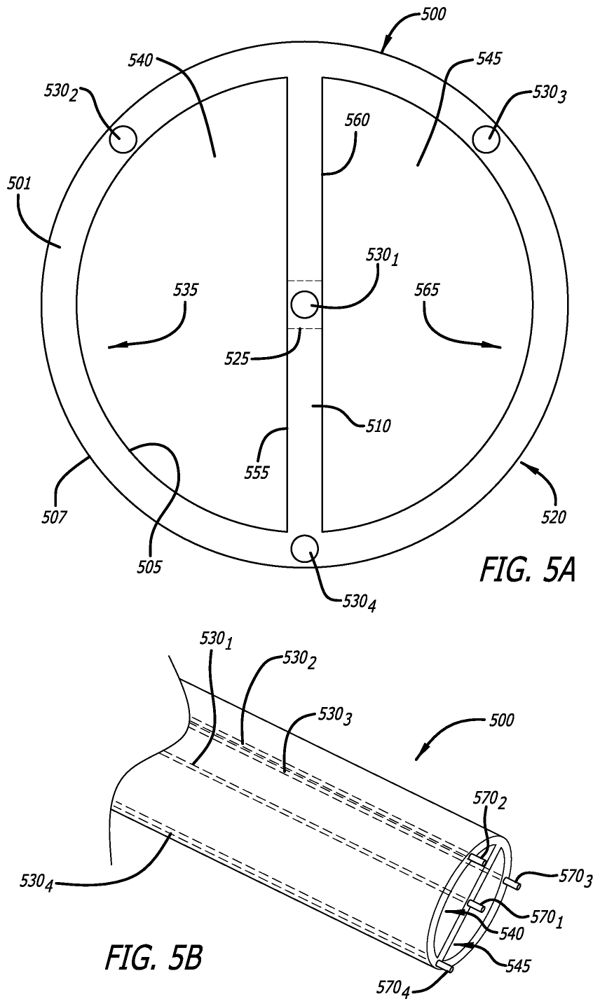

Referring to FIG. 5A, an elevation view of an illustrative embodiment of an elongate probe (e.g., the elongate probe 120) in the form of a catheter 500 including integrated tubing, a diametrically disposed septum, and micro-lumens formed within the tubing and septum is shown in accordance with some embodiments. Herein, the catheter 500 includes integrated tubing, the diametrically disposed septum 510, and the plurality of micro-lumens 530₁-530₄ which, for this embodiment, are fabricated to reside within the wall 501 of the integrated tubing of the catheter 500 and within the septum 510. In particular, the septum 510 separates a single lumen, formed by the inner surface 505 of the wall 501 of the catheter 500, into multiple lumen, namely two lumens 540 and 545 as shown. Herein, the first lumen 540 is formed between a first arc-shaped portion 535 of the inner surface 505 of the wall 501 forming the catheter 500 and a first outer surface 555 of the septum 510 extending longitudinally within the catheter 500. The second lumen 545 is formed between a second arc-shaped portion 565 of the inner surface 505 of the wall 501 forming the catheter 500 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 500 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 501 of the catheter 500. In particular, a second micro-lumen 530₂ is fabricated within the wall 501 of the catheter 500, namely between the inner surface 505 and outer surface 507 of the first arc-shaped portion 535 of the wall 501. Similarly, the third micro-lumen 530₃ is also fabricated within the wall 501 of the catheter 500, namely between the inner and outer surfaces 505/507 of the second arc-shaped portion 555 of the wall 501. The fourth micro-lumen 530₄ is also fabricated within the inner and outer surfaces 505/507 of the wall 501 that are aligned with the septum 510.

According to one embodiment of the disclosure, as shown in FIG. 5A, the micro-lumens 5302-5304 are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 500 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 501.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000 μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 500 itself. Similarly, the first micro-lumen 530₁ may be sized to retain the center core fiber 570₁, where the diameter of the first micro-lumen 530₁ may be sized just larger than the diameter of the center core fiber 5701.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

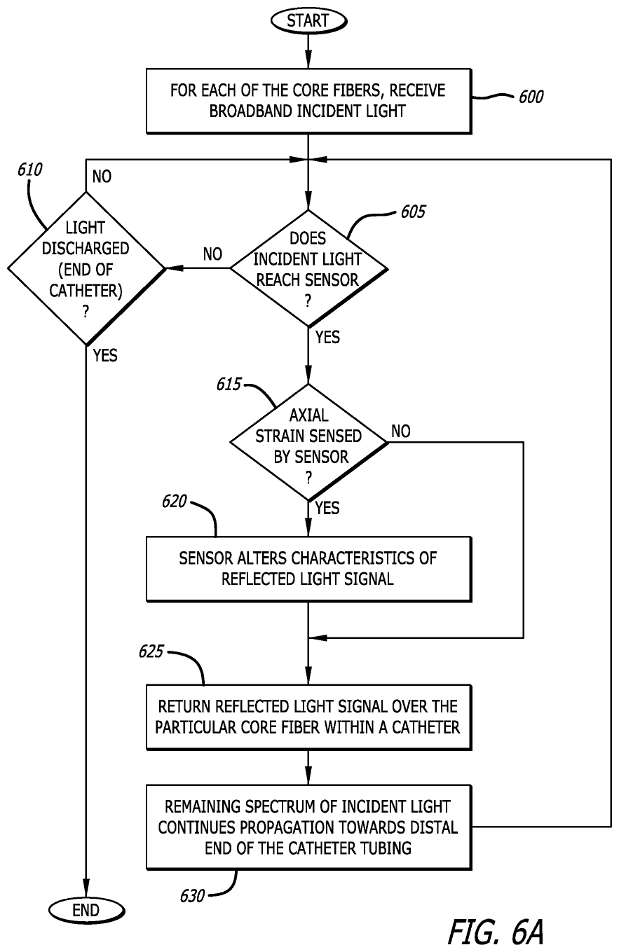

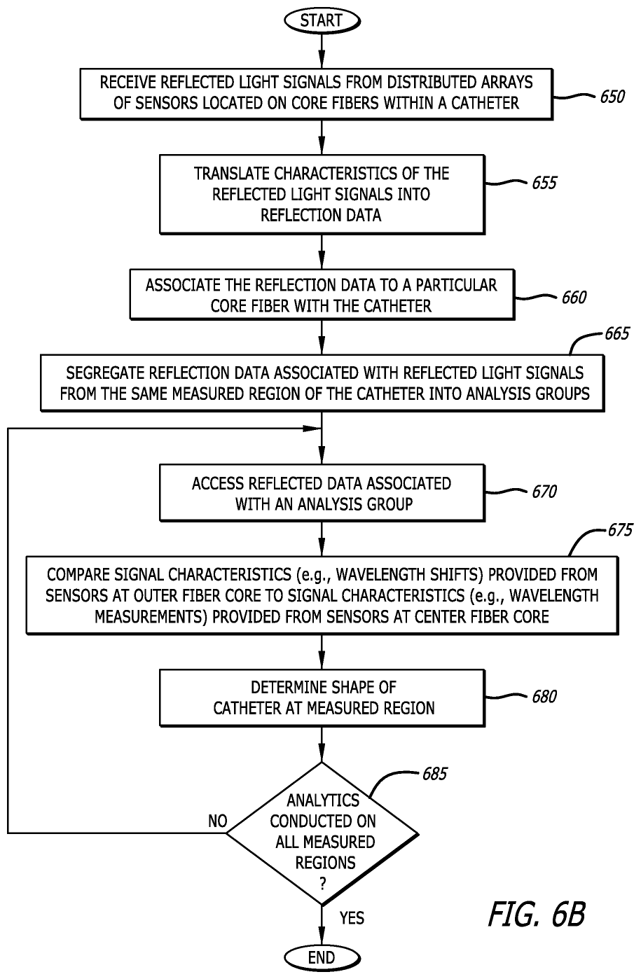

Referring to FIGS. 6A-6B, flowcharts of methods of operations conducted by the medical instrument monitoring system of FIG. 1 to achieve optic three-dimensional shape sensing are shown in accordance with some embodiments. Herein, an elongate probe in the form of a catheter such as the catheter 500 of FIGS. 5A-5B, includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From this analytics, on all analytic groups (e.g., reflected light signals from sensors in all or most of the core fibers), the shape sensing logic may determine the shape the core fibers have taken in three-dimensional space, from which the shape sensing logic can determine the current physical state of the catheter in three-dimension space (blocks 680-685).

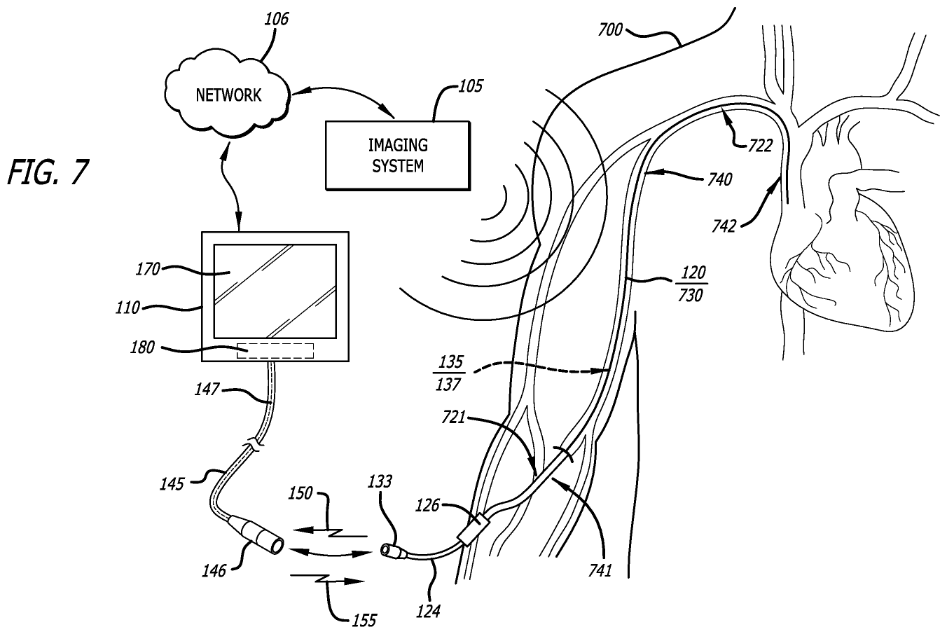

FIG. 7 illustrates one implementation of the system 100 of FIG. 1, where an elongate probe of a medical instrument is inserted into a vasculature of a patient, in accordance with some embodiments. Herein, the elongate probe 120 generally includes a proximal portion 721 that remains exterior to the patient 700 and a distal portion 722 that resides within the patient vasculature after placement is complete. The elongate probe 120 may be advanced along a course 740 within the patient 700 (e.g., a series of vessels such as veins or arteries). In the illustrated embodiment, the course 740 is defined by the vasculature of the patient 700 extending between an insertion site 741 and a distal point, such as the Superior Vena Cava (“SVC”) 742.

During advancement along the course 740, the elongate probe 120 receives broadband incident light 155 from the console 110 via optical fiber(s) 147 within the interconnect 145, where the incident light 155 propagates along the core fibers 137 of the multi-core optical fiber 135 within the elongate probe 120. According to one embodiment of the disclosure, the connector 146 of the interconnect 145 terminating the optical fiber(s) 147 may be coupled to the optical-based console connector 133, which may be configured to terminate the core fibers 137 deployed within the elongate probe 120. Such coupling optically connects the core fibers 137 of the elongate probe 120 with the optical fiber(s) 147 within the interconnect 145. The optical connectivity is needed to propagate the incident light 155 to the core fibers 137 and return the reflected light signals 150 to the optical logic 180 within the console 110 over the interconnect 145. Further during advancement along the course 740, the shape sensing logic 194 determines the physical state of the optical fiber 135 (or more specifically the 3D shape of the optical fiber 135) which defines a live 3D shape 730 of the elongate probe 120. In some embodiments, various instruments may be disposed at the distal end 122 of the probe 120 to measure a fluid pressure within the body (e.g., blood pressure in a certain heart chamber and/or in the blood vessels), measure a fluid (e.g., blood) flow rate, measure a temperature, view an interior of the body via a camera, or the like.

In some embodiments, an imaging system 105 may be communicatively coupled with the console 110 via the network 106. In use, the console 110 may receive image data from the image system 105 for processing by the shape pathway logic 195. The image data may include an image of at least a portion of the elongate probe 120 advanced along the course 740 in combination with anatomical elements, e.g., the heart, veins, etc. as shown and further described below.

During advancement of the elongate probe 120, the live 3D shape 730 assumes various 3D shapes, such as generally straight portions and/or curved portions in accordance with the shape of the course 740. During advancement, known conditions of the live 3D shape 730 at one location may facilitate predicting the live 3D shape 730 at a subsequent location. In a similar fashion, known conditions of the patient anatomy may also facilitate predicting the live 3D shape 730. For example, in the illustrated embodiment, known conditions for the course 740 extending along the vasculature from the insertion site 741 to the SVC 742 may include a relatively straight proximal portion of the live 3D shape 730 extending along an arm of the patient followed by a curved distal portion extending into the SVC 742. Other known conditions may include physiologic conditions, such as an ECG signal near the SVC 742, for example.

In some embodiments, the known conditions may include a construction or structure of the elongate probe 120. For example, the elongate probe 120 may be a stent insertion device having a pre-formed shape configured for advancement along an arterial vasculature. In such an example, the pre-formed shape may be a known condition for defining a pathway for the live 3D shape 730. In some embodiments, actions of the elongate probe 120 may provide known conditions. By way of another example, the elongate probe 120 may be a steerable drainage catheter. In such an example, the action of steering the catheter during advancement may be employed in defining the pathway and/or the buffer zone.

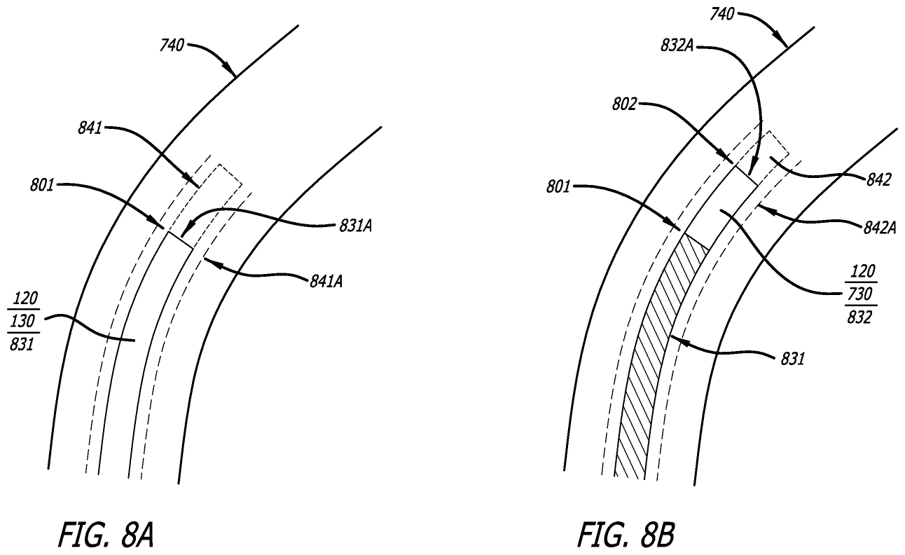

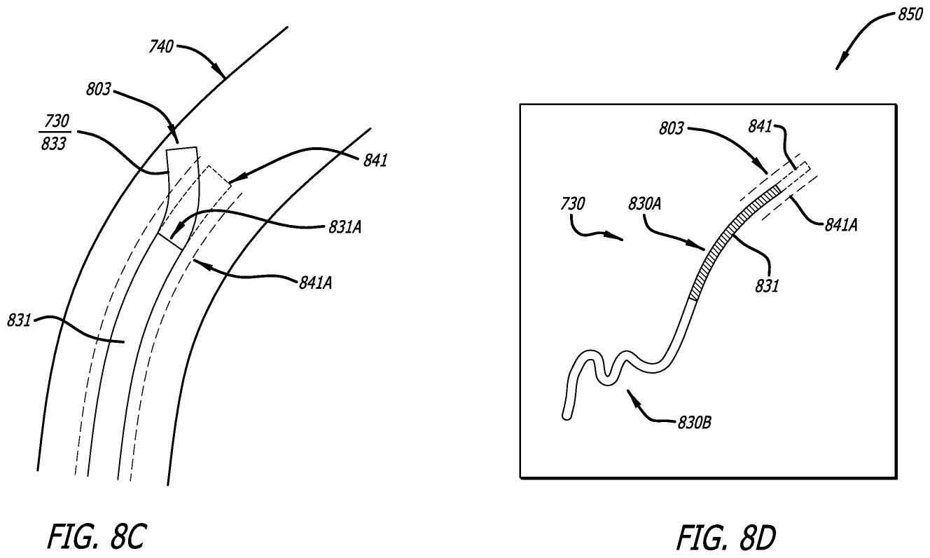

In some embodiments, in addition to generating the live 3D shape 730, the shape sensing logic 194 may be also generate a visual representation of the live 3D shape 730 that is configured to be displayed on the display screen 170 (or on another physical display screen, such as that of a tablet or other network device). An illustrative example of such is shown in FIG. 8D.

FIGS. 8A-8B illustrate the live 3D shape 730 at sequential states of insertion of the elongate probe 120 along the course 740. FIG. 8A illustrates the elongate probe 120 together with the live 3D shape 730 at a first location 801 (defined by the location of a distal tip of the probe 120). The shape pathway logic 195 captures a first reference shape 831 of the live 3D shape 730 at the first location 801 and defines a pathway 841 extending distally away from a distal end 831A of the first reference shape 831. The shape pathway logic 195 may also define a buffer zone 841A extending radially outward of the pathway 841.

FIG. 8B illustrates the live 3D shape 730 at a second location 802, where the probe 120 is advanced distally in relation to the first location 801 of the FIG. 8A. As illustrated, the live 3D shape 730 follows the pathway 841 during advancement from the first location 801 to the second location 802. With the live 3D shape 730 advanced to the second location 802, the shape pathway logic 195 captures a second reference shape 832 of the live 3D shape 730, and defines a pathway 842 extending distally away from the distal end 832A of the second reference shape 832. The shape pathway logic 195 also defines a buffer zone 842A extending radially outward of the pathway 842.

During advancement of the probe 120 along the course 740, the shape pathway logic 195 may repeatedly (i) capture a reference shape of the live 3D shape 730, (ii) define a pathway extending distally away from the distal end of the reference shape, and (iii) define a buffer zone extending radially outward of the pathway. In some embodiments, the buffer zone may extend proximally along at least a portion of the reference shape 842.

FIG. 8C illustrates an exemplary instance during insertion of the probe in contrast to the state of insertion of FIG. 8B. In some instances, the probe 120 may not advance along the course 740 as intended. In such an instance, the live 3D shape 730 may not follow (or align with) the pathway 841. Shown is the live 3D shape 730 at a third location 803, where the live 3D shape 730 is advanced distally in relation to the first location 801. At the third location 803, the live 3D shape 730 deviates from the pathway 841 sufficiently to exceed the buffer zone 841A. In such an instance, the shape pathway logic 195 may compare the live 3D shape 730 with the buffer zone 841A, and as a result of the comparison, the shape pathway logic 195 may provide notification to the clinician that a portion of the live 3D shape 730 exceeds the buffer zone 841A.

As further illustrated in FIG. 8C, in some instances, a current reference shape may deviate from a preceding reference shape beyond a threshold amount. In the illustrated instance, the current reference shape 833 does not align with, i.e., is inconsistent with, the preceding reference shape 831. In such an instance, the shape pathway logic 195 may compare the current reference shape 833 with the preceding reference shape 831, and as a result of the comparison, determine that the current reference shape 833 is an invalid reference shape. In such an instance, the shape pathway logic 195 may prevent the reference shape 833 from being used to further define the pathway for the live 3D shape 730.

In some embodiments, a rules-based system may be used in which, based on the preceding reference shape 831, a threshold boundary is established circumferentially around the preceding reference shape 831. Thus, when the current reference shape 833 is determined to be outside of the threshold boundary in any direction, the shape pathway logic 195 determines the probe 120 has moved in an unexpected or undesired manner.

In some instances, the threshold boundary may be established based on calculations from the preceding reference shape 831 (e.g., the current reference shape 833 may only move a percentage X in any circumferential direction, e.g., 10%, in order for the pathway logic 195 to consider the current reference shape 833 valid).

In some instances, the threshold boundary may be established through the use of machine learning techniques. For example, a machine learning model may be developed to provide output indicating the probability of a valid current reference shape based at least on the preceding reference shape. Stated otherwise, a machine learning model may be trained on historical data of pathways through patient vasculatures (e.g., labeled as valid for supervised training or unlabeled for unsupervised) such that the trained machine learning model takes as input data corresponding to the preceding reference shape and provides probabilities for valid subsequent reference shapes. The threshold boundary may then be determined based on the resultant probabilities (e.g., the threshold boundary may utilize positionings with at least Y percent probability of being valid, e.g., 95%).

FIG. 8D is an exemplary screen shot of the live 3D shape. The screen shot 850 renders image of the live 3D shape 730 at a state of insertion, such as the state shown in FIG. 8A, for example. In the illustrated screen shot 850, a portion 830A of the live 3D shape 730 may be inserted within the patient and a portion 830B may reside outside the patient. In some embodiments, the screen shot 850 may render an image of the pathway 841 extending distally in front of the reference shape 831, of which an image may also be rendered. In some embodiments, the screen shot 850 may also render an image of the buffer zone 841A.

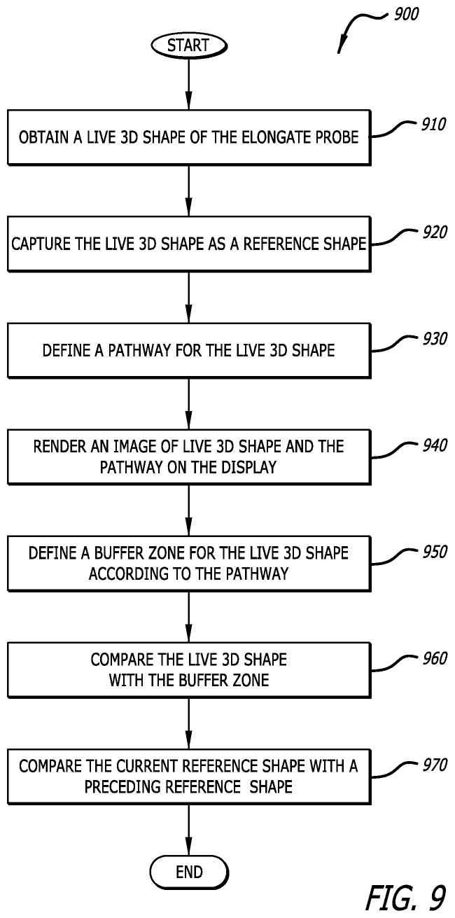

FIG. 9 illustrates a flowchart of a method of operations as may be performed by the medical instrument monitoring system of FIG. 1 to define a pathway for the live 3D shape of the elongate probe, in accordance with some embodiments. The method 900 may be performed by the shape pathway logic 195. In other embodiments, the shape pathway logic 195 may be incorporated into the shape sensing logic 194, and as such, the method 900 may be performed by the shape sensing logic 194. The method 900 generally processes shape data to generate a pathway for the live 3D shape to follow during insertion. According to one embodiment of the disclosure, as shown in FIG. 9, the shape pathway logic 195 obtains the live shape 3D shape of the elongate probe (block 910). More specifically, the shape sensing logic 194 determines the live 3D shape and provides shape data pertaining to the live 3D shape to the shape pathway logic 195.

The shape pathway logic 195 then captures a snapshot of the live 3D shape as a reference shape (block 920). The reference shape becomes a record in memory of the live 3D shape at the point in time when the live 3D shape was captured.

The shape pathway logic 195 then defines a pathway for the live 3D shape to follow during further insertion (block 930). The shape pathway logic 195 defines the pathway in accordance with the reference shape. The shape pathway logic 195 may also define the pathway according other known conditions related to the elongate probe and/or the patient as discussed above. The shape pathway logic 195 may also define the pathway according to a plurality of reference shapes including one or more reference shapes captured during insertion of preceding elongate probes.

With the pathway defined, the shape pathway logic 195 may render an image of the pathway on the display together with image of the live 3D shape (block 940). In some instances, the clinician may be familiar with previous valid 3D shapes, and rendering an image of the live 3D shape together with the pathway projected in front of the live 3D shape provides an opportunity for the clinician to assess the pathway in relation to 3D shapes from previous elongate probes.

The shape pathway logic 195 may also define a buffer zone extending radially away from the pathway (block 950). The shape pathway logic 195 may define a buffer zone buffer as a limit for displacement of the live 3D shape in relation to the pathway. The buffer zone may also extend proximally along the reference shape. During insertion and/or use of the elongate probe, the shape pathway logic 195 may compare the live 3D shape with the buffer zone, and as a result of the comparison, the shape pathway logic 195 may provide an alert or other notification to the clinician when a portion of the live 3D shape exceeds the buffer zone (block 960).

In some embodiments, the shape pathway logic 195 may compare a current reference shape with one or more preceding reference shapes (block 970). The shape pathway logic 195 may capture and store a plurality of reference shapes in memory and then compare a current reference shape with the preceding reference shapes to determine a validity of the current reference shape. For example, if a current reference shape aligns with one or more preceding valid reference shapes stored in memory, the shape pathway logic 195 may deem the current reference shape as valid and store the current reference shape in a data base of valid reference shapes. By way of contrast, if a current reference shape deviates from one or more preceding valid reference shapes stored in memory, the shape pathway logic 195 may deem the current reference shape as invalid.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device system, comprising:
a medical device comprising:
an elongate probe; and
an optical fiber having one or more of core fibers extending along the elongate probe, each of the one or more core fibers including a plurality of sensors distributed along the longitudinal length and each sensor of the plurality of sensors being configured to (i) reflect a light signal of a different spectral width based on received incident light, and (ii) change a characteristic of the reflected light signal based on strain experienced by the optical fiber; and
a console including one or more processors and a non-transitory computer-readable medium having stored thereon logic, when executed by the one or more processors, causes operations including:
determining a live three-dimensional (3D) shape of the elongate probe during insertion of the elongate probe within a patient body, wherein determining includes:
providing an incident light signal to the optical fiber;
receiving reflected light signals of different spectral widths of the incident light by one or more of the plurality of sensors; and
processing the reflected light signals associated with the one or more of core fibers to determine the live 3D shape;
capturing a reference shape, the reference shape including at least a portion of the live 3D shape; and
defining a pathway for the live 3D shape, the pathway extending distally away from a distal end of the reference shape.

2. The system according to claim 1, wherein the medical device is one of an intravascular device, an endoscope, a biopsy device, a drainage catheter, a surgery device, a tissue ablation device, or a kidney stone removal device.

3. The system according to claim 1, wherein the operations further include rendering an image of the reference shape on a display of the console.

4. The system according to claim 1, wherein the operations further include rendering an image of the pathway on the display.

5. The system according to claim 1, wherein the operations further include rendering an image of the live 3D shape in combination with the image of the pathway on the display.

6. The system according to claim 1, wherein the operations further include:
comparing the live 3D shape with the reference shape; and
as a result of the comparison, detecting an insertion and/or withdrawal displacement of the elongate probe.

7. The system according to claim 1, wherein the operations further include:
capturing a plurality of reference shapes of the live 3D shape, and
defining the pathway in accordance with the plurality of reference shapes.

8. The system according to claim 1, wherein the operations further include:
defining a buffer zone for the live 3D shape, the buffer zone extending radially away from the pathway;
comparing the live 3D shape with the buffer zone; and
as a result of the comparison, providing a notification when a portion of the live 3D shape exceeds the buffer zone.

9. The system according to claim 1, wherein:
the system is communicatively coupled with an imaging system, and
the operations further include:
receiving image data from the imaging system; and
defining the pathway in accordance with the image data.

10. The system according to claim 9, wherein the imaging system includes one or more of an ultrasound imaging system, a magnetic resonance imaging (MRI) system, a computed tomography (CT) imaging system, an X-ray system, an X-ray system including fluoroscopy, or an electro-anatomical mapping system.

11. The system according to claim 1, wherein:
the elongate probe includes one or more sensors configured to detect physiological conditions of the patient, and
the operations further include defining the pathway in accordance with sensor data pertaining to the physiological conditions.

12. The system according to claim 11, wherein the physiological conditions include one or more of a body temperature, a fluid pressure, a blood flow rate, or an ECG signal.

13. The system according to claim 1, wherein the operations further include defining the pathway in accordance with one or more reference shapes captured during insertion of previous elongate probes.

14. A method for detecting placement of a medical device within a patient body, the method comprising:
providing the medical device coupled with a medical device system, the medical device including an elongate probe configured for insertion within the patient body:
determining a live three-dimensional (3D) shape of the elongate probe inserted within the patient body, wherein determining includes:
providing an incident light signal to an optical fiber extending along the elongate probe, wherein the optical fiber includes a one or more of core fibers, each of the one or more of core fibers including a plurality of reflective gratings distributed along a longitudinal length of a corresponding core fiber and each of the plurality of reflective gratings being configured to (i) reflect a light signal of a different spectral width based on received incident light, and (ii) change a characteristic of the reflected light signal based on strain experienced by the optical fiber;
receiving reflected light signals of different spectral widths of the incident light by one or more of the plurality of sensors; and
processing the reflected light signals associated with the one or more of core fibers to determine the three-dimensional shape of the elongate probe inserted within the patient body;
capturing a reference shape, the reference shape including at least a portion of the live 3D shape; and
defining a pathway for the live 3D shape, the pathway extending distally away from a distal end of the reference shape.

15. The method according to claim 14, further comprising rendering an image of the reference shape on a display of the medical device system.

16. The method according to claim 14, further comprising rendering an image of the pathway on the display.

17. The method according to claim 14, further comprising rendering an image of the live 3D shape in combination with the image of the pathway on the display.

18. The method according to claim 14, further comprising:
comparing the live 3D shape with the reference shape; and
as a result of the comparison, detecting an insertion and/or withdrawal displacement of the elongate probe.

19. The method according to claim 14, further comprising:
capturing a plurality of reference shapes of the live 3D shape, and
defining the pathway in accordance with the plurality of reference shapes.

20. The method according to claim 14, further comprising:
defining a buffer zone for the live 3D shape, the buffer zone extending radially away from the pathway;
comparing the live 3D shape with the buffer zone; and
as a result of the comparison, providing a notification when a portion of the live 3D shape exceeds the buffer zone.

21. The method according to claim 14, further comprising:
coupling the medical device system with an imaging system;
receiving image data from the imaging system; and
defining the pathway in accordance with the image data.

22. The method according to claim 21, wherein the imaging system includes one or more of an ultrasound imaging system, a magnetic resonance imaging (MRI) system, a computed tomography (CT) imaging system, an X-ray system, an X-ray system including fluoroscopy, or an electro-anatomical mapping system.

23. The method according to claim 14, wherein the elongate probe includes one or more sensors configured to detect physiological conditions of the patient, the method further comprising defining the pathway in accordance with sensor data pertaining to the physiological conditions.

24. The method according to claim 23, wherein the physiological conditions include one or more of a body temperature, a blood pressure, a blood flow rate, or an ECG signal.

25. The method according to claim 14, further comprising defining the pathway in accordance with one or more reference shapes captured during insertion of previous elongate probes.
